(19) United States
(12) Patent Application Publication
(10) Pub. No.: US 2018/0271190 A1
(43) Pub. Date: Sep. 27, 2018

(54) Wearable Shirt With Component Parts for Medical Access

(71) Applicant: Robert Campbell, Kailua-Kona, HI (US)

(72) Inventor: Robert Campbell, Kailua-Kona, HI (US)

(73) Assignee: Robert Cambell, Kailua-Kona, HI (US)

(21) Appl. No.: 15/469,567

(22) Filed: Mar. 26, 2017

(51) Int. Cl. A41D 13/12; A41D 27/20; A61M 5/142

(52) U.S. Cl. CPC A41D 13/1245; A61M 5/14244; A41D 27/20

(57) ABSTRACT

A medical access shirt having a medical access configuration and a non-medial access configuration. The medical access shirt includes a shirt body having a pair of front shirt panels and a back shirt panel with the pair of front shirt panels coupled to the back shirt panel. The at least one of the front shirt panels includes a chest panel and an abdominal panel, the chest panel separable from the abdominal panel and the other front shirt panel. The shirt body is operable to transition the medical access shirt between the medical access configuration and the non-medical access configuration by separating the chest panel from the abdominal panel.

### TECHNICAL FIELD OF THE DISCLOSURE

This disclosure relates, in general, to medical shirts worn by individuals requiring ongoing medical treatment and, in particular, to a shirt that includes component parts that discretely stores a treatment facilitator and provides easy access to the individual for delivering ongoing medical treatments from the facilitator.

### BACKGROUND

Individuals inflicted with a disease may need continuous medical treatment. In some cases, a patient with serious diseases, such as cancer, may require intravenous treatments and these treatments may be ongoing over a period of months if not years. Traditionally, in order for medical practitioners to administer necessary treatments, the patient must disrobe or at least partially disrobe to allow the practitioner to clean an infusion port and insert intravenous tubing. Typically in these situations, the patient may seek treatment at inpatient or outpatient treatment facilities. As such, the patient when entering these facilities may be robed in traditional clothing and specific clothing associated with the area around the infusion port may need to be removed. For some individuals, particularly woman, this may be considered a little intrusive and, as such, a little uncomfortable. In addition, often these treatments require periodic facilitation of a therapeutic drug or drug regimen over a period of hours, days, or weeks. In these cases, this requires an infusion pump to administer the drug intravenously. Conventionally, the pump is often indiscreetly worn around the patient's waist, e.g. using what would be called a fanny pack.

As such, a medical access shirt that can be worn outside of the treatment facility in a traditional manner that can function to also provide easy access to an infusion port and discrete storage of an infusion pump is needed.

### SUMMARY

The disclosure is generally directed to a medical access shirt which includes component parts that discretely stores a treatment facilitator and provides easy access to the individual for delivering ongoing treatment from the facilitator. An illustrative embodiment of the medical access shirt includes a shirt body having a pair of front shirt panels and a back shirt panel with the pair of front shirt panels coupled to the back shirt panel. The shirt body further includes at least one of the front shirt panels having a chest panel and an abdominal panel. The chest panel may be separable from the abdominal panel and the other front shirt panel. The shirt body being operable to transition the medical access shirt between the medical access configuration and the non-medical access configuration by separating the chest panel from the abdominal panel.

In some embodiments, the medical access shirt may include at least one of the panels having a storage receptacle for receiving an infusion pump. The storage receptacle may be coupled with the abdominal panel. Alternatively, the storage receptacle may be coupled with the chest panel or the other chest panel.

These and other aspects, objects, features and advantages of the example embodiments will become apparent to those having ordinary skill in the art upon consideration of the following detailed description of illustrated example embodiments.

### BRIEF DESCRIPTION OF THE DRAWINGS

For a more complete understanding of the features and advantages of the present disclosure, reference is now made to the detailed description along with the accompanying figures in which corresponding numerals in the different figures refer to corresponding parts and in which:

FIG. 1 is a front view of a wearable shirt in a non-medical access configuration according to an embodiment of the invention;

FIGS. 2A and 2B are front views of the wearable shirt in a first medical access configuration and a second medical access configuration according to an embodiment of the invention;

FIG. 3 is a back view of the wearable shirt that illustrates an alternative configuration for a chest panel according to an embodiment of the invention; and

FIG. 4 is a front view of the wearable shirt that includes a support strap according to an embodiment of the invention.

### DETAILED DESCRIPTION

While various systems, methods and other embodiments are discussed in detail below, it should be appreciated that the present disclosure provides many applicable inventive concepts, which can be embodied in a wide variety of specific contexts. The specific embodiments discussed herein are merely illustrative and do not delimit the scope of the present disclosure.

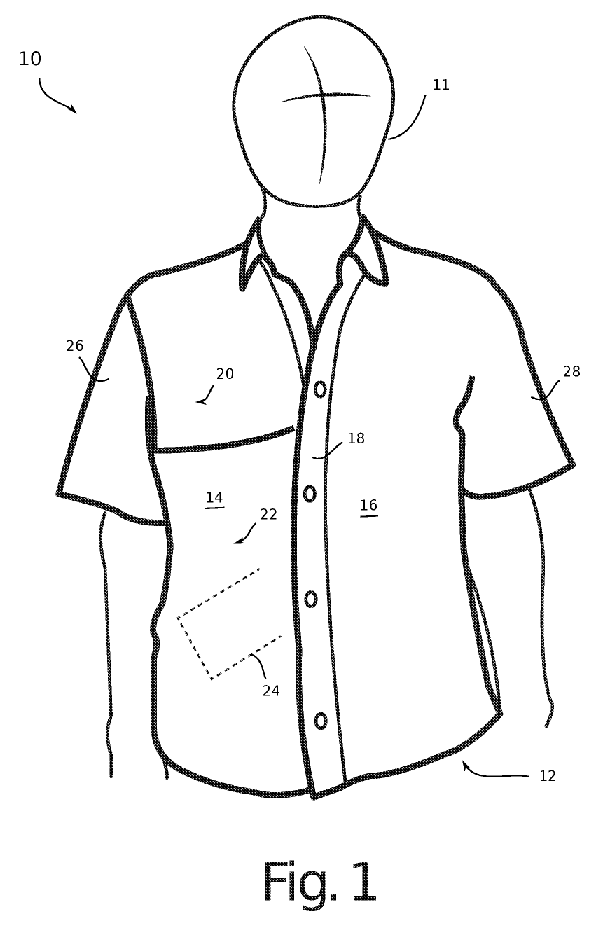

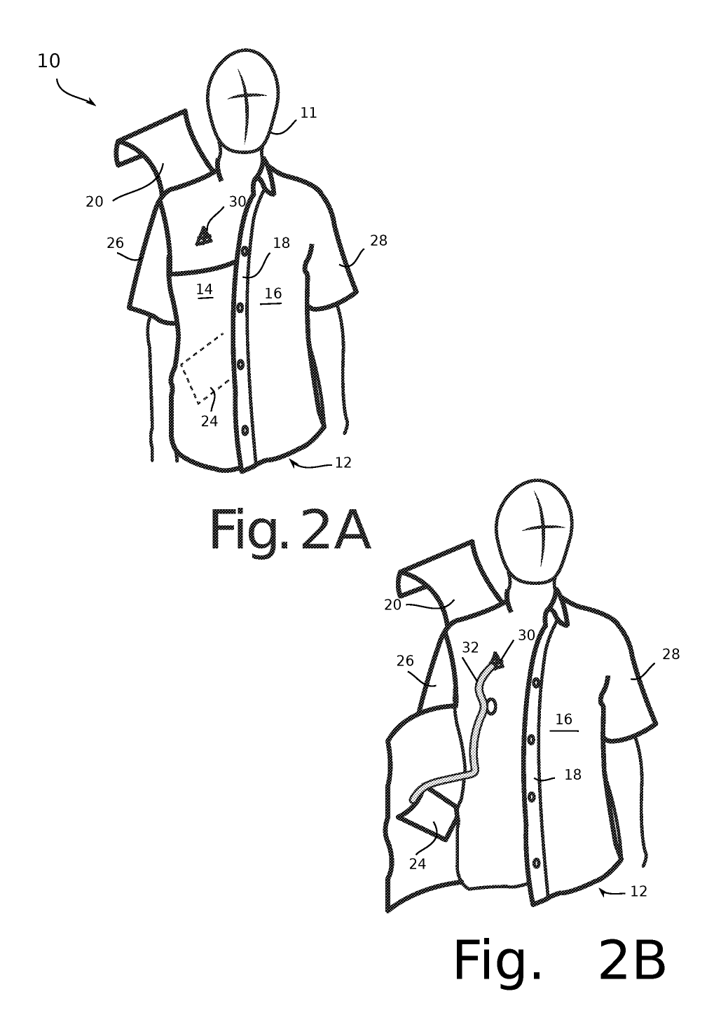

Referring to FIG. 1, illustrative is a shirt having component parts for facilitating medical access according to an embodiment of the invention and is denoted generally as 10. The shirt 10 may be worn by a medical patient 11, or stated differently a wearer, and includes a shirt body 12 made from cotton, polyester, cotton/polyester blend or other material that is consistent with the functional requirements of the shirt 10. The shirt body 12 may include a pair of front shirt panels 14, 16 and a back panel, not illustrated in FIG. 1. A generally elongated, vertical front shirt opening 18 may separate or divide the front shirt panels 14, 16. Shirt panel 14, or alternatively shirt panel 16, may include a chest panel 20 and an abdominal panel 22. The chest panel 20 may be separable from and couplable with the abdominal panel 22, e.g. using a fastener, VELCRO, or even an adhesive. The abdominal panel 22 further includes a storage receptacle 24, such as pocket sown into the inner lining of the shirt 10. The shirt 10 may further include sleeves 26, 28 sown into the seams of the back panel and abdominal panel 22. The chest panel 20 may also be separable from and couplable with the sleeves 26, 28. The chest panel 20 when separated from the abdominal panel 22, as best illustrated in FIG. 2A, allows access to the upper torso of the wearer 11 which allows medical professionals to insert an infusion port 30 into the anatomy of the wearer 11 and further allows medical professionals or the wearer 11 to clean the infusion port 30. When the abdominal panel 22 is separated from the the chest panel 20 and the other panel 16, or optionally the opening 18 or optionally the opening 18 and the panel 16, as best illustrated in FIG. 2B, it further exposes the anatomy of the wearer 11 allowing medical personal to clean the infusion port 30, remove or install an infusion pump in the receptacle 24, and couple or decouple the infusion pump with intravenous tubing 32 used to couple the pump to the infusion port 30. When the shirt 10 is assembled so that the chest panel 20 is coupled with the abdominal panel 22, and possibly, with the other shirt panel 16 and possible with the the opening 18 or possibly within only the shirt panel 16 or opening 18, the shirt 10 can be worn in a traditional manner. The benefits of such is that it allows the wearer 11 to discretely store and comfortably carry therapeutic facilitators.

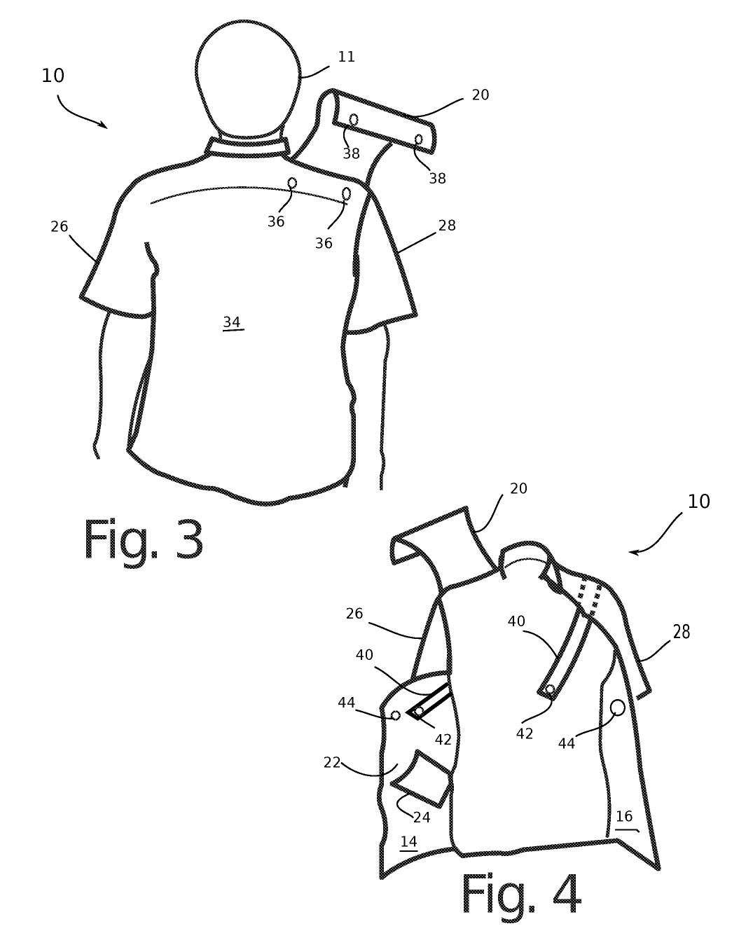

Referring now to FIG. 3, illustrated is an embodiment of shirt 10 that includes back panel 34 and chest panel 20, not illustrated in FIG. 3, than can be worn by wearer 11. In this particular embodiment, shirt 10 may include sleeves 26, 28. In this particular embodiment, shirt 10 may further include male fasteners 36 and female fastener 38 positioned on back panel 34 and chest panel 20 strategically placed in a way that allows the chest panel 20 to be completely detached, as opposed to being partially detached, from shirt panels 14,16, shirt opening 18, and sleeves 26, 28. It should be understood, however, that the male and female fasteners 36, 38 may be configured in an opposite way. The benefit of such is that the wearer 11 may completely remove chest panel 20 of the shirt 10 in a way that can assist medical professionals in doing necessary work without unnecessary hindrance and provide the wearer 11 with a level of comfort needed during these times.

Referring now to FIG. 4, illustrated is another embodiment of the shirt 10 that includes supportive features that may further increase the wearers 11 comfort level. In this particular embodiment, shirt 10 may include chest panel 20, abdominal panel 22, storage receptacle 24, and sleeves 26, 28. In this particular embodiment, shirt 10 may further include a support strap 40. Support strap further includes male fasteners 42 coupled with support strap 40 and female fasteners 44 coupled with shirt panels 14, 16. Male fasteners 42 and female fasteners capable of being couple together. Although, it should be understood that the male fasteners 42 and female fasteners 44 may be coupled to strap 42 and shirt panels 14, 16 in an opposite configuration.

It should be understood by those skilled in the art that the illustrative embodiments described herein are not intended to be construed in a limiting sense. Various modifications and combinations of the illustrative embodiments as well as other embodiments will be apparent to persons skilled in the art upon reference to this disclosure. It is, therefore, intended that the appended claims encompass any such modifications or embodiments.

What is claimed is:

1. A medical access shirt having a medical access configuration and a non-medial access configuration, comprising:
a shirt body having a pair of front shirt panels and a back shirt panel with the pair of front shirt panels coupled to the back shirt panel, with at least one of the front shirt panels including a chest panel and an abdominal panel, the chest panel separable from the abdominal panel and the other front shirt panel;
wherein, the shirt body is operable to transition the medical access shirt between the non-medical access configuration and the medical access configuration by separating the chest panel from the abdominal panel.

2. The medical access shirt as recited in claim 1 wherein the at least one of the panels includes a storage receptacle for receiving an infusion pump.

3. The medical access shirt as recited in claim 2 wherein the storage receptacle is coupled with the abdominal panel.

4. The medical access shirt as recited in claim 2 wherein the storage receptacle is coupled with the chest panel.

5. The medical access shirt as recited in claim 2 wherein the storage receptacle is coupled with the other shirt panel.

6. The medical access shirt as recited in claim 2 wherein the storage receptacle is coupled with the back shirt panel.

7. The medical access shirt as recited in claim 1 wherein the shirt body includes sleeves.

8. The medical access shirt as recited in claim 1 wherein the chest panel, abdominal panel, and other panel are securely couplable together using a mechanical fastener.

9. The medical access shirt as recited in claim 1 wherein the chest panel, abdominal panel, and other panel are securely coupleable together using an adhesive.

10. The medical access shirt as recited in claim 1 wherein the chest panel, abdominal panel, and other panel are securely couplable together using Velcro.

11. A medical access shirt having a medical access configuration and a non-medial access configuration, comprising:
a shirt body having a pair of front shirt panels and a back shirt panel with the pair of front shirt panels coupled to the back shirt panel, with at least one of the front shirt panels including a chest panel and an abdominal panel, the chest panel separable from the abdominal panel and the other front shirt panel; and
a storage receptacle for receiving an infusion pump coupled to at least one of the front shirt panels and the back shirt panels;
wherein, the shirt body is operable to transition the medical access shirt between the medical access configuration and the non-medical access configuration by separating the chest panel from the abdominal panel.

12. The medical access shirt as recited in claim 11 wherein the storage receptacle is coupled with the abdominal panel.

13. The medical access shirt as recited in claim 12 wherein the storage receptacle is coupled with the chest panel.

14. The medical access shirt as recited in claim 12 wherein the storage receptacle is coupled with the other shirt panel.

15. The medical access shirt as recited in claim 12 wherein the storage receptacle is coupled with the back shirt panel.
